(19) United States
(12) Patent Application Publication
(10) Pub. No.: US 2022/0233344 A1
(43) Pub. Date: Jul. 28, 2022

(54) Targeted Temperature Management Systems, Pads, and Methods Thereof for Treating Burn Wounds

(71) Applicant: C. R. Bard, Inc., Franklin Lakes, NJ (US)

(72) Inventor: Michael R. Hoglund, Windsor, CO (US)

(21) Appl. No.: 17/584,101

(22) Filed: Jan. 25, 2022

Related U.S. Application Data
(60) Provisional application No. 63/141,801, filed on Jan. 26, 2021.

(51) Int. Cl. A61F 7/00; A61F 13/02; A61F 7/08

(52) U.S. Cl. CPC A61F 7/0085; A61F 2013/00157; A61F 7/08; A61F 13/0223

(57) ABSTRACT

Disclosed herein are targeted temperature management (“TTM”) systems, pads, and methods thereof for treating burn wounds. A method of a system for TTM can include a pad-connecting step, a pad-placing step, and a fluid-circulating step. The pad-connecting step can include connecting an inlet and an outlet of a pad to a hydraulic system of a control module. The pad can include a multilayered pad body having a conduit layer configured to convey a temperature-controlled fluid provided by the control module. The pad-placing step can include placing the pad on a wounded portion of a patient's body with a sterile, thermally conductive wound-healing layer of the pad body in contact with a burn wound of the wounded portion of the patient's body. The fluid-circulating step can include circulating the temperature-controlled fluid through the conduit layer to cool the wounded portion of the patient's body, thereby treating the wound to promote healing.

### PRIORITY

This application claims the benefit of priority to U.S. Provisional Patent Application No. 63/141,801, filed Jan. 26, 2021, which is incorporated by reference in its entirety into this application.

### BACKGROUND

Targeted temperature management (“TTM”) is a treatment for maintaining a therapeutic body temperature in a patient for a period of time to improve the patient's outcome in any of a number of different medical situations. Current TTM systems and methods require TTM pads be adhered to patients during TTM to maintain sufficient contact between the TTM pads and the patients while circulating temperature-controlled fluid (e.g., cooled fluid) through the TTM pads. Due to the requirement of current TTM systems and methods to adhere the TTM pads to the patients, however, TTM is clearly not an indicated treatment for burn victims and their burn wounds—despite the potential of TTM to cool tissues of such burn wounds and mitigate any further tissue damage. Indeed, adhering the TTM pads to the burn victims could aggravate their burn wounds instead of helping them heal. What is needed then are TTM systems, pads, and methods thereof for treating the foregoing burn victims and their burn wounds to promote healing.

Disclosed herein are TTM systems, pads, and methods thereof for treating burn victims and their wounds to promote healing.

### SUMMARY

Disclosed herein is a pad for TTM including, in some embodiments, a multilayered pad body and a backing over the pad body. The pad body includes a conduit layer, an impermeable film over the conduit layer, and a sterile, thermally conductive wound-healing layer over the impermeable film. The conduit layer includes one or more conduits configured to convey a fluid through the conduit layer. The impermeable film is configured to retain the fluid in the conduit layer when the fluid is conveyed through the conduit layer. The wound-healing layer is configured for placement on a wounded portion of a patient's body. The backing is over the wound-healing layer in a ready-to-use state of the pad. The backing is configured to maintain sterility of at least the wound-healing layer prior to use of the pad.

In some embodiments, the wound-healing layer includes a hydrogel. The hydrogel is selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the wound-healing layer is chemically stable to sterilization via autoclave, ethanol, or ultraviolet light.

In some embodiments, the wound-healing layer is configured for healing a burn wound. The burn wound is selected from a heat burn, a friction burn, an electrical burn, a radiation burn, and frostbite.

In some embodiments, the wound-healing layer includes one or more antimicrobial agents. The one-or-more antimicrobial agents are selected from an aminoglycoside including neomycin, kanamycin, gentamycin, or tobramycin; a cyclic peptide including polymyxin B or polymyxin E; a sulfonamide including mafenide acetate or silver sulfadiazine; a tetracycline including doxycycline or minocycline; a cationic steroid antimicrobial (“CSA”) including CSA-8; a metal-based antimicrobial including an organometallic compound including silver sulfadiazine, an elemental metal including a preparation of silver or copper nanoparticles, a metal oxide including a preparation of zinc-oxide nanoparticles, or a salt including silver nitrate, a Cu(II) salt, or a Ga(III) salt; a halogen-based antimicrobial including a diatomic halogen molecule including iodine, a halophor including iodopovidone, or a salt including sodium hypochlorite; chlorhexidine; vancomycin; fusidic acid; usnic acid; bacitracin; mupirocin; nitrofurazone; nystatin; rifampicin; and a biofilm-disrupting agent including a 2-aminobenzimidazole or a D-amino acid.

In some embodiments, the pad further includes an inlet. The inlet is configured for charging the conduit layer with the fluid.

In some embodiments, the pad further includes an outlet. The outlet is configured for discharging the fluid from the conduit layer.

Also disclosed is a system for TTM including, in some embodiments, a control module and pad. The control module includes a hydraulic system. The hydraulic system includes a chiller evaporator, a heater, one or more outlets, and one or more inlets. The chiller evaporator is configured for fluid cooling. The heater is configured for fluid heating. Together, the chiller evaporator and the heater are configured to provide a temperature-controlled fluid. The one-or-more outlets are configured for discharging the temperature-controlled fluid as a supply fluid from the hydraulic system. The one-or-more inlets are configured for charging the hydraulic system with a return fluid to continue to produce the temperature-controlled fluid. The pad includes a multilayered pad body and a backing over the pad body. The pad body includes a conduit layer, an impermeable film over the conduit layer, and a sterile, thermally conductive wound-healing layer over the impermeable film. The conduit layer includes one or more conduits configured to convey the supply fluid through the conduit layer. The impermeable film is configured to retain the supply fluid in the conduit layer when the supply fluid is conveyed through the conduit layer. The wound-healing layer is configured for placement on a wounded portion of a patient's body. The backing is over the wound-healing layer in a ready-to-use state of the pad. The backing is configured to maintain sterility of at least the wound-healing layer prior to use of the pad.

In some embodiments, the wound-healing layer of the pad includes a hydrogel. The hydrogel is selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the wound-healing layer of the pad is chemically stable to sterilization via autoclave, ethanol, or ultraviolet light.

In some embodiments, the wound-healing layer is configured for healing a burn wound. The burn wound is selected from a heat burn, a friction burn, an electrical burn, a radiation burn, and frostbite.

In some embodiments, the wound-healing layer of the pad includes one or more antimicrobial agents. The one-or-more antimicrobial agents are selected from an aminoglycoside including neomycin, kanamycin, gentamycin, or tobramycin; a cyclic peptide including polymyxin B or polymyxin E; a sulfonamide including mafenide acetate or silver sulfadiazine; a tetracycline including doxycycline or minocycline; a cationic steroid antimicrobial (“CSA”) including CSA-8; a metal-based antimicrobial including an organometallic compound including silver sulfadiazine, an elemental metal including a preparation of silver or copper nanoparticles, a metal oxide including a preparation of zinc-oxide nanoparticles, or a salt including silver nitrate, a Cu(II) salt, or a Ga(III) salt; a halogen-based antimicrobial including a diatomic halogen molecule including iodine, a halophor including iodopovidone, or a salt including sodium hypochlorite; chlorhexidine; vancomycin; fusidic acid; usnic acid; bacitracin; mupirocin; nitrofurazone; nystatin; rifampicin; and a biofilm-disrupting agent including a 2-aminobenzimidazole or a D-amino acid.

In some embodiments, the pad further includes an inlet. The inlet is configured for charging the conduit layer with the supply fluid.

In some embodiments, the pad further includes an outlet. The outlet is configured for discharging the return fluid from the conduit layer.

Also disclosed herein is a method of a system for TTM. The method includes a pad-connecting step, a pad-placing step, and a fluid-circulating step. The pad-connecting step includes connecting an inlet and an outlet of a pad to a hydraulic system of a control module. The pad includes a multilayered pad body having a conduit layer configured to convey a temperature-controlled fluid provided by the control module. The pad-placing step includes placing the pad on a wounded portion of a patient's body with a sterile, thermally conductive wound-healing layer of the pad body in contact with a wound of the wounded portion of the patient's body. The fluid-circulating step includes circulating the temperature-controlled fluid through the conduit layer to cool or warm the wounded portion of the patient's body, thereby treating the wound to promote healing.

In some embodiments, the method further includes a backing-removal step. The backing-removal step includes removing a backing of the pad to reveal the wound-healing layer before the pad-placing step. The backing is configured to maintain sterility of at least the wound-healing layer prior to using the pad.

In some embodiments, the fluid-circulating step transfers heat between the temperature-controlled fluid and the wounded portion of the patient's body by thermal conduction through the wound-healing layer. The wound-healing layer includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, treating the wound to promote healing further includes the pad-placing step as the wound-healing layer includes one or more antimicrobial agents for synergistically mitigating microbial growth about the wound.

In some embodiments, the one-or-more antimicrobial agents are selected from an aminoglycoside including neomycin, kanamycin, gentamycin, or tobramycin; a cyclic peptide including polymyxin B or polymyxin E; a sulfonamide including mafenide acetate or silver sulfadiazine; a tetracycline including doxycycline or minocycline; a cationic steroid antimicrobial (“CSA”) including CSA-8; a metal-based antimicrobial including an organometallic compound including silver sulfadiazine, an elemental metal including a preparation of silver or copper nanoparticles, a metal oxide including a preparation of zinc-oxide nanoparticles, or a salt including silver nitrate, a Cu(II) salt, or a Ga(III) salt; a halogen-based antimicrobial including a diatomic halogen molecule including iodine, a halophor including iodopovidone, or a salt including sodium hypochlorite; chlorhexidine; vancomycin; fusidic acid; usnic acid; bacitracin; mupirocin; nitrofurazone; nystatin; rifampicin; and a biofilm-disrupting agent including a 2-aminobenzimidazole or a D-amino acid.

In some embodiments, the fluid-circulating step includes circulating a cool fluid through the conduit layer for treating the wound. The wound is selected from a heat burn, a friction burn, an electrical burn, and a radiation burn in such embodiments.

In some embodiments, the fluid-circulating step includes circulating a warm fluid through the conduit layer for treating the wound. The wound being frostbite in such embodiments.

These and other features of the concepts provided herein will become more apparent to those of skill in the art in view of the accompanying drawings and following description, which describe particular embodiments of such concepts in greater detail.

### DRAWINGS

FIG. 1 illustrates a TTM system in accordance with some embodiments.

FIG. 2A illustrates TTM pads for a torso of a patient in accordance with some embodiments.

FIG. 2B illustrates TTM pads for legs of a patient in accordance with some embodiments.

FIG. 3 illustrates a multilayered pad body of a TTM pad in accordance with some embodiments.

FIG. 4 illustrates a hydraulic system of a control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, current TTM systems and methods require TTM pads be adhered to patients during TTM to maintain sufficient contact between the TTM pads and the patients while circulating temperature-controlled fluid (e.g., cooled fluid) through the TTM pads. Due to the requirement of current TTM systems and methods to adhere the TTM pads to the patients, however, TTM is clearly not an indicated treatment for burn victims and their burn wounds—despite the potential of TTM to cool tissues of such burn wounds and mitigate any further tissue damage. Indeed, adhering the TTM pads to the burn victims could aggravate their burn wounds instead of helping them heal. What is needed then are TTM systems, pads, and methods thereof for treating the foregoing burn victims and their burn wounds to promote healing.

Disclosed herein are TTM systems, pads, and methods thereof for treating burn victims and their wounds to promote healing. That said, the TTM systems, pads, and methods set forth herein are not limited to burn victims and their burn wounds. Indeed, other trauma victims and their wounds (e.g., lacerations, abrasions, skin infections, etc.) can also benefit from the TTM systems, pads, and methods set forth herein.

### TTM Systems

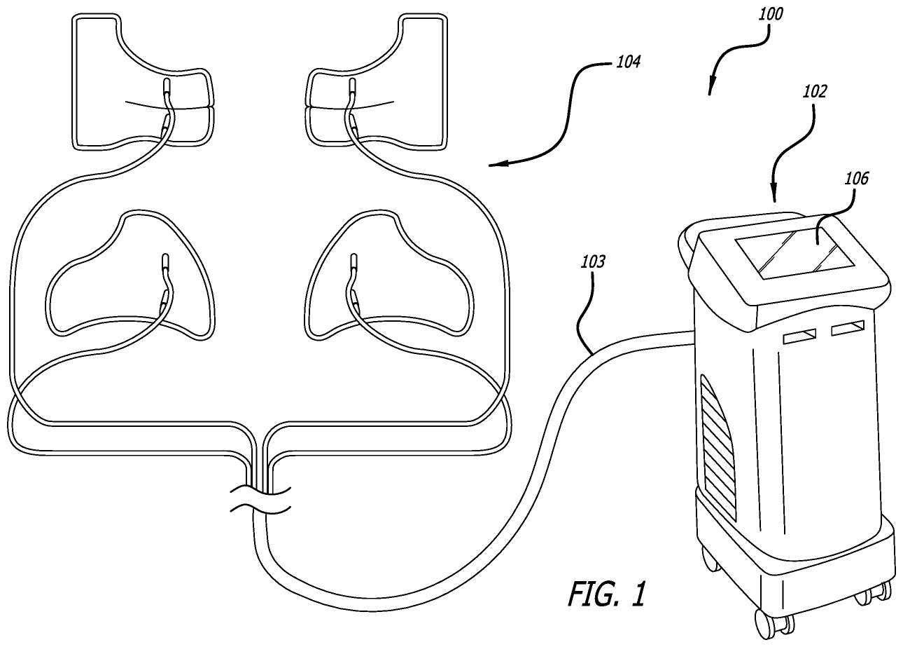

FIG. 1 illustrates a TTM system 100 in accordance with some embodiments.

As shown, the system 100 can include a control module 102, one or more TTM pads 104 such as those set forth below, and one or more fluid conduits 103 therebetween. Description for the control module 102 is set forth immediately below. Description for the one-or-more pads 104 is set forth in the following section.

The control module 102 can include a console 106 with an integrated display screen configured as a touchscreen for operating the control module 102. The console 106 can include one or more processors, primary and secondary memory, and instructions stored in the primary memory configured to instantiate one or more processes for TTM with the control module 102.

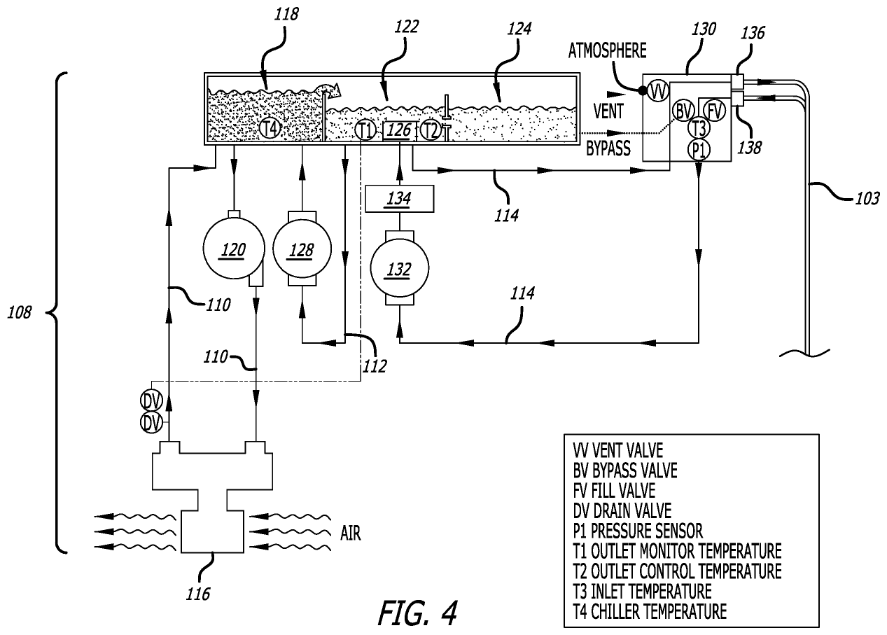

FIG. 4 illustrates a hydraulic system 108 of the control module 102 in accordance with some embodiments.

The control module 102 can also include the hydraulic system 108, which can include a chiller circuit 110, a mixing circuit 112, and a circulating circuit 114.

The chiller circuit 110 can be configured for cooling a fluid (e.g., water, ethylene glycol, a combination of water and ethylene glycol, etc.) to produce a cooled fluid, which cooled fluid, in turn, can be for mixing with the mixed fluid in the mixing tank 122 set forth below to produce a supply fluid for TTM. The chiller circuit 110 can include a chiller evaporator 116 configured for the cooling of the fluid passing therethrough. The fluid for the cooling by the chiller evaporator 116 is provided by a chiller tank 118 using a chiller pump 120 of the chiller circuit 110.

The mixing circuit 112 can be configured for mixing spillover of the cooled fluid from the chiller tank 118 with a mixed fluid in a mixing tank 122 of the mixing circuit 112. The mixing circuit 112 can include a heater 126 in the mixing tank 122 configured for heating the mixed fluid to produce a heated fluid if needed for mixing with the cooled fluid to provide a supply tank 124 of the circulating circuit 114 with the supply fluid of a desired temperature for TTM. The mixing circuit 112 can include a mixing pump 128 configured to pump the fluid from the mixing tank 122 into the chiller tank 118 for producing the cooled fluid as well as the spillover of the cooled fluid for the mixing tank 122.

The circulating circuit 114 can be configured for circulating the supply fluid for TTM, which includes circulating the supply fluid provided by the manifold 130 through the one-or-more pads 104 using a circulation pump 132 directly or indirectly governed by a flow meter 134 of the circulating circuit 114. The manifold 130 can include one or more outlets 136 configured for discharging the supply fluid (e.g., a cooled fluid or a warmed fluid as indicated) from the hydraulic system 108 and one or more inlets 138 configured for charging the hydraulic system 108 with return fluid from the one-or-more pads 104 to continue to produce the supply fluid.

A thermoelectric TTM system is an alternative to the system 100 set forth above. The thermoelectric system can include a control module, one or more thermoelectric pads, and one or more electrical cables therebetween. Like the control module 102, the control module of the thermoelectric system can include one or more processors, primary and secondary memory, and instructions stored in the primary memory configured to instantiate one or more processes for thermoelectric TTM with the control module. Notably, the control module of the thermoelectric system need not have a hydraulic system.

### TTM Pads

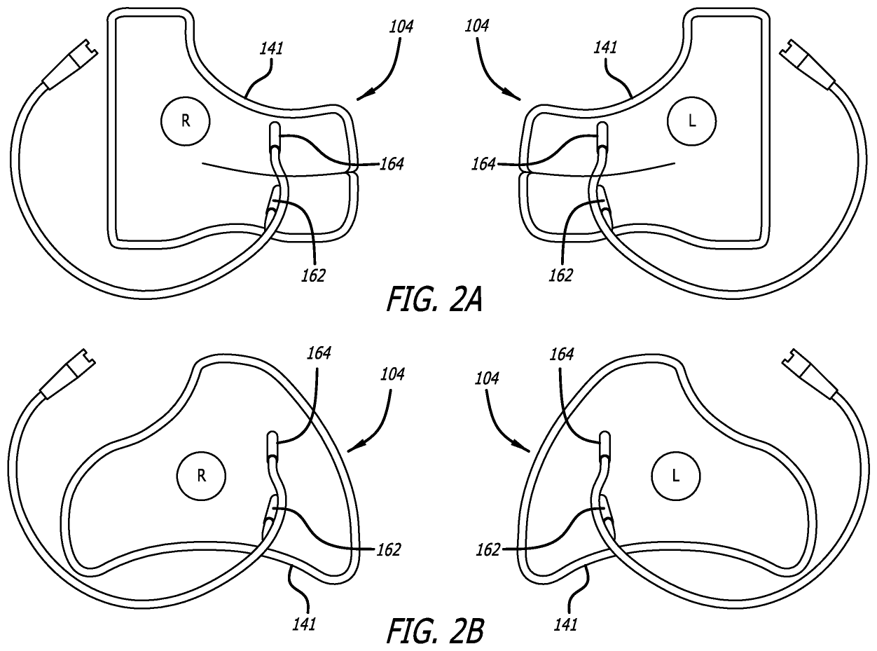

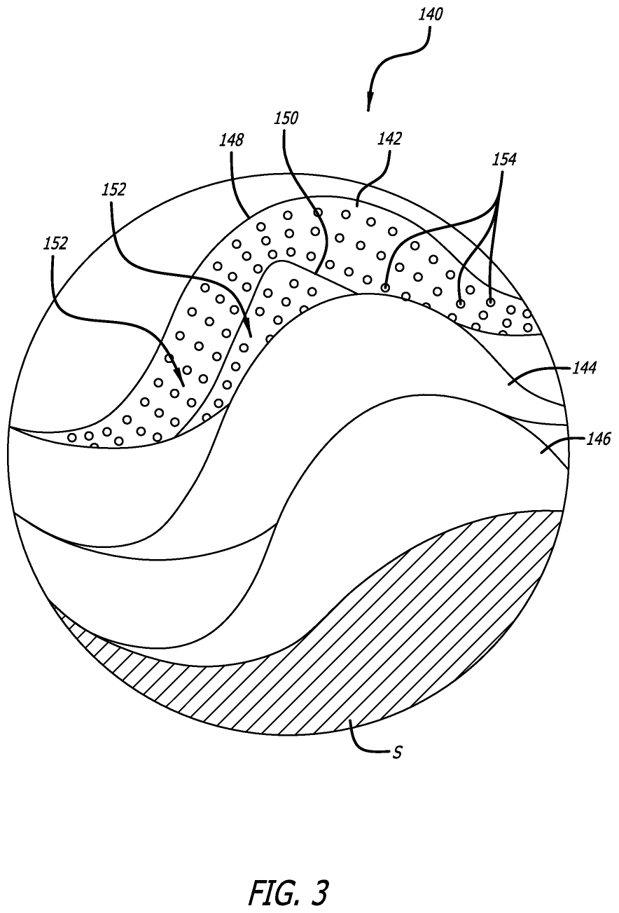

FIGS. 2A and 2B illustrate left and right pads of the one-or-more pads 104 respectively for a torso and legs of a patient in accordance with some embodiments. FIG. 3 illustrates a multilayered pad body 140 of a pad of the-one-or-more pads 104 in accordance with some embodiments.

A pad of the one-or-more pads 104 can include the pad body 140 and a backing 141 over the pad body 140.

The pad body 140 can include a conduit layer 142, an impermeable film 144 over the conduit layer 142, and a sterile, thermally conductive wound-healing layer 146 over the impermeable film 144.

The conduit layer 142 includes a perimetrical wall 148 and one or more inner walls 150 extending from the conduit layer 142 toward the impermeable film 144. Together, the perimetrical wall 148 and the one-or-more inner walls 150 form one or more conduits 152 configured to convey the supply fluid through the conduit layer 142.

The conduit layer 142 can include a plurality of protrusions 154 extending from the conduit layer 142 toward the impermeable film 144. The protrusions 154 are configured to promote even flow of the supply fluid when the supply fluid is conveyed through the conduit layer 142.

The conduit layer 142 can be of an insulating foam. The insulating foam can be configured to prevent heat loss into an ambient environment.

The impermeable film 144 can be configured to retain the supply fluid in the conduit layer 142 when the supply fluid is conveyed through the conduit layer 142. In addition, the impermeable film 144 can be configured to allow efficient energy transfer between the conduit layer 142 and the wound-healing layer 146.

The wound-healing layer 146 can be configured for placement on skin S of wounded portion (e.g., torso, leg, etc.) of a patient's body for direct thermal conduction through the wound-healing layer 146. While the wound-healing layer 146 can be configured to conformably adhere to the wounded portion of the patient's body for better thermal conduction, adherence of the wound-healing layer 146 to the wounded portion of the patient's body can be optimized to avoid further wounding the wounded portion of the patient's body upon removal of the pad.

The wound-healing layer 146 can be configured for healing a burn wound. The burn wound can be one requiring cooling to mitigate further tissue damage such as a heat burn, a friction burn (e.g., a heat burn from an abrasion such as from sliding across the ground in a motorcycle accident), an electrical burn, or a radiation burn (e.g., a sunburn) Alternatively, the burn wound can be one requiring warming to mitigate further tissue damage such as frostbite. However, as set forth above, the wound-healing layer 146 can be configured for healing wounds other than burn wounds such as lacerations, abrasions, skin infections, or the like.

The wound-healing layer 146 can include a hydrogel or hydrogel matrix. The hydrogel can be selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

The wound-healing layer 146 can be chemically stable to terminal sterilization by a physical or chemical method of sterilization such as sterilization by heat (e.g., steam heat), radiation (e.g., non-ionizing radiation such as ultraviolet light, ionizing radiation such as gamma radiation, etc.), a liquid (e.g., an alcohol such as ethyl alcohol, isopropyl alcohol, etc.), or a gas (e.g., an electrophile such as ethylene oxide, an oxidant such as hydrogen peroxide, ozone, etc.). For example, the wound-healing layer 146 can be chemically stable to terminal sterilization by autoclave, one or more washes with ethanol, or irradiation with ultraviolet light.

The wound-healing layer 146 can include one or more therapeutic agents to synergistically treat the wounded portion of the patient's body and promote healing. The one-or-more therapeutic agents can include one-or-more antimicrobial agents (e.g., antibacterial agents, antifungal agents, etc.). The one-or-more antimicrobial agents can be selected from an aminoglycoside such as neomycin, kanamycin, gentamycin, or tobramycin; a cyclic peptide such as polymyxin B or polymyxin E; a sulfonamide such as mafenide acetate or silver sulfadiazine; a tetracycline such as doxycycline or minocycline; a cationic steroid antimicrobial (“CSA”) such as CSA-8; a metal-based antimicrobial including an organometallic compound such as silver sulfadiazine, an elemental metal such as a preparation of silver or copper nanoparticles, a metal oxide such as a preparation of zinc-oxide nanoparticles, or a salt such as silver nitrate, a Cu(II) salt, or a Ga(III) salt; a halogen-based antimicrobial including a diatomic halogen molecule such as iodine, a halophor such as iodopovidone, or a salt such as sodium hypochlorite; a biofilm-disrupting agent including a 2-aminobenzimidazole or a D-amino acid; chlorhexidine; vancomycin; fusidic acid; usnic acid; bacitracin; mupirocin; nitrofurazone; nystatin; and rifampicin. For example, the one-or-more antimicrobial can be a combination of neomycin, bacitracin, and polymyxin B.

Advantageously, delivery rates of certain therapeutic agents of the one-or-more therapeutic agents can be modulated by way of temperature of the supply fluid to either increase or decrease the rates of delivery to the wounded portion of the patient's body.

The backing 141 can be over the wound-healing layer 146 in a ready-to-use state of the pad. The backing 141 is configured to maintain sterility of at least the wound-healing layer 146 prior to use of the pad.

In addition to the foregoing, the pad can include an inlet 162 and an outlet 164. The inlet 162 is configured for charging the conduit layer 142 with the supply fluid, while the outlet 164 is configured for discharging the return fluid from the conduit layer 142.

As set forth above, the alternative thermoelectric TTM system includes the one-or-more thermoelectric pads. Because TTM is not effectuated with the supply fluid provided by the control module 102, the one-or-more thermoelectric pads need not have the conduit layer 142; instead, the one-or-more thermoelectric pads can include a thermoelectric layer including one or more thermoelectric devices (e.g., an array of thermoelectric devices). The one-or-more thermoelectric devices can be configured to undergo a temperature change upon application of a voltage difference across the one-or-more thermoelectric devices.

### Methods

Methods of the systems and pads include methods of use. For example, a method of using the system 100 can include a backing-removal step, a pad-connecting step, a pad-placing step, and a fluid-circulating step.

The backing-removal step can include removing the backing 141 of a pad of the one-or-more pads 104 to reveal the wound-healing layer 146. As set forth above, the backing 141 can be configured to maintain sterility of at least the wound-healing layer 146 prior to using the pad.

The pad-connecting step can include respectively connecting the inlet 162 and the outlet 164 of the pad to an outlet of the one-or-more outlets 136 and an inlet of the one-or-more inlets 138 of the hydraulic system 108 of the control module 102. The pad-connecting step can further include connecting one or more other pads of the one-or-more pads 104 to the hydraulic system 108 in parallel with the foregoing pad.

The pad-placing step can include placing the pad on a wounded portion (e.g., torso, leg, etc.) of a patient's body with the sterile, thermally conductive wound-healing layer 146 of the pad body 140 in contact with a wound of the wounded portion of the patient's body.

The fluid-circulating step can include circulating a temperature-controlled fluid (i.e., the supply fluid) provided by the control module 102 through the conduit layer 142 to cool or warm the wounded portion of the patient's body, thereby treating the wound to promote healing. The fluid-circulating step can transfer heat between the temperature-controlled fluid and the wounded portion of the patient's body by thermal conduction through the wound-healing layer 146, which, as set forth above, includes a hydrogel or hydrogel matrix. The fluid-circulating step can include circulating a cool fluid as the temperature-controlled fluid through the conduit layer 142 for treating the wound if it requires cooling to mitigate tissue damage. In such embodiments, the wound can be a heat burn, a friction burn, an electrical burn, or a radiation burn. Alternatively, the fluid-circulating step can include circulating a warm fluid as the temperature-controlled fluid through the conduit layer 142 for treating the wound if it requires warming to mitigate tissue damage. In such embodiments, the wound can be frostbite.

In an alternative to the fluid-circulating step, the method can include a voltage-applying step when using the thermoelectric TTM system. The voltage applying step includes applying by way of the control module of the thermoelectric TTM system a difference in voltage across the one-or-more thermoelectric devices to effectuate TTM with the one-or-more thermoelectric pads.

Treating the wound to promote healing can further include the pad-placing step set forth above. Indeed, placing the pad on the wounded portion of the patient's body with the wound-healing layer 146 in contact with the wound thereof can result in administering to the wound of the wounded portion of the patient's body the one-or-more antimicrobial agents of the wound-healing layer 146 for synergistically mitigating microbial growth about the wound.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A pad for targeted temperature management (“TTM”), comprising:
a multilayered pad body including:
a conduit layer including one or more conduits configured to convey a fluid through the conduit layer;
an impermeable film over the conduit layer configured to retain the fluid in the conduit layer when the fluid is conveyed through the conduit layer; and
a sterile, thermally conductive wound-healing layer over the impermeable film configured for placement on a wounded portion of a patient's body; and
a backing over the wound-healing layer in a ready-to-use state of the pad, the backing configured to maintain sterility of at least the wound-healing layer prior to use of the pad.

2. The pad of claim 1, wherein the wound-healing layer includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

3. The pad of claim 1, wherein the wound-healing layer is chemically stable to sterilization via autoclave, ethanol, or ultraviolet light.

4. The pad of claim 1, wherein the wound-healing layer is configured for healing a burn wound selected from a heat burn, a friction burn, an electrical burn, a radiation burn, and frostbite.

5. The pad of claim 1, wherein the wound-healing layer includes one or more antimicrobial agents selected from an aminoglycoside including neomycin, kanamycin, gentamycin, or tobramycin; a cyclic peptide including polymyxin B or polymyxin E; a sulfonamide including mafenide acetate or silver sulfadiazine; a tetracycline including doxycycline or minocycline; a cationic steroid antimicrobial (“CSA”) including CSA-8; a metal-based antimicrobial including an organometallic compound including silver sulfadiazine, an elemental metal including a preparation of silver or copper nanoparticles, a metal oxide including a preparation of zinc-oxide nanoparticles, or a salt including silver nitrate, a Cu(II) salt, or a Ga(III) salt; a halogen-based antimicrobial including a diatomic halogen molecule including iodine, a halophor including iodopovidone, or a salt including sodium hypochlorite; chlorhexidine; vancomycin; fusidic acid; usnic acid; bacitracin; mupirocin; nitrofurazone; nystatin; rifampicin; and a biofilm-disrupting agent including a 2-aminobenzimidazole or a D-amino acid.

6. The pad of claim 1, further comprising an inlet configured for charging the conduit layer with the fluid.

7. The pad of claim 6, further comprising an outlet configured for discharging the fluid from the conduit layer.

8. A system for targeted temperature management (“TTM”), comprising:
a control module including a hydraulic system having:
a chiller evaporator configured for fluid cooling;
a heater configured for fluid heating, the chiller evaporator and the heater, together, configured to provide a temperature-controlled fluid;
one or more outlets configured for discharging the temperature-controlled as a supply fluid from the hydraulic system; and
one or more inlets configured for charging the hydraulic system with a return fluid to continue to produce the temperature-controlled fluid; and
a pad including:
multilayered pad body including:
a conduit layer including one or more conduits configured to convey the supply fluid through the conduit layer;
an impermeable film over the conduit layer configured to retain the supply fluid in the conduit layer when the supply fluid is conveyed through the conduit layer; and
a sterile, thermally conductive wound-healing layer over the impermeable film configured for placement on a wounded portion of a patient's body; and
a backing over the wound-healing layer in a ready-to-use state of the pad, the backing configured to maintain sterility of at least the wound-healing layer prior to use of the pad.

9. The system of claim 8, wherein the wound-healing layer of the pad includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

10. The system of claim 8, wherein the wound-healing layer of the pad is chemically stable to sterilization via autoclave, ethanol, or ultraviolet light.

11. The system of claim 8, wherein the wound-healing layer is configured for healing a burn wound selected from a heat burn, a friction burn, an electrical burn, a radiation burn, and frostbite.

12. The system of claim 8, wherein the wound-healing layer of the pad includes one or more antimicrobial agents selected from an aminoglycoside including neomycin, kanamycin, gentamycin, or tobramycin; a cyclic peptide including polymyxin B or polymyxin E; a sulfonamide including mafenide acetate or silver sulfadiazine; a tetracycline including doxycycline or minocycline; a cationic steroid antimicrobial (“CSA”) including CSA-8; a metal-based antimicrobial including an organometallic compound including silver sulfadiazine, an elemental metal including a preparation of silver or copper nanoparticles, a metal oxide including a preparation of zinc-oxide nanoparticles, or a salt including silver nitrate, a Cu(II) salt, or a Ga(III) salt; a halogen-based antimicrobial including a diatomic halogen molecule including iodine, a halophor including iodopovidone, or a salt including sodium hypochlorite; chlorhexidine; vancomycin; fusidic acid; usnic acid; bacitracin; mupirocin; nitrofurazone; nystatin; rifampicin; and a biofilm-disrupting agent including a 2-aminobenzimidazole or a D-amino acid.

13. The system of claim 8, the pad further comprising an inlet configured for charging the conduit layer with the supply fluid.

14. The system of claim 13, the pad further comprising an outlet configured for discharging the return fluid from the conduit layer.

15. A method of a system for targeted temperature management (“TTM”), comprising:
connecting an inlet and an outlet of a pad to a hydraulic system of a control module, the pad including a multilayered pad body having a conduit layer configured to convey a temperature-controlled fluid provided by the control module;
placing the pad on a wounded portion of a patient's body with a sterile, thermally conductive wound-healing layer of the pad body in contact with a wound of the wounded portion of the patient's body; and
circulating the temperature-controlled fluid through the conduit layer to cool or warm the wounded portion of the patient's body, thereby treating the wound to promote healing.

16. The method of claim 15, further comprising removing a backing of the pad to reveal the wound-healing layer before placing the pad on the wounded portion of the patient's body, the backing configured to maintain sterility of at least the wound-healing layer prior to using the pad.

17. The method of claim 15, wherein circulating the temperature-controlled fluid through the conduit layer transfers heat between the temperature-controlled fluid and the wounded portion of the patient's body by thermal conduction through the wound-healing layer, the wound-healing layer including a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogel s.

18. The method of claim 15, wherein treating the wound to promote healing further includes the placing of the pad on the wounded portion of a patient's body, the wound-healing layer including one or more antimicrobial agents for synergistically mitigating microbial growth about the wound.

19. The method of claim 18, wherein the one-or-more antimicrobial agents are selected from an aminoglycoside including neomycin, kanamycin, gentamycin, or tobramycin; a cyclic peptide including polymyxin B or polymyxin E; a sulfonamide including mafenide acetate or silver sulfadiazine; a tetracycline including doxycycline or minocycline; a cationic steroid antimicrobial (“CSA”) including CSA-8; a metal-based antimicrobial including an organometallic compound including silver sulfadiazine, an elemental metal including a preparation of silver or copper nanoparticles, a metal oxide including a preparation of zinc-oxide nanoparticles, or a salt including silver nitrate, a Cu(II) salt, or a Ga(III) salt; a halogen-based antimicrobial including a diatomic halogen molecule including iodine, a halophor including iodopovidone, or a salt including sodium hypochlorite; chlorhexidine; vancomycin; fusidic acid; usnic acid; bacitracin; mupirocin; nitrofurazone; nystatin; rifampicin; and a biofilm-disrupting agent including a 2-aminobenzimidazole or a D-amino acid.

20. The method of claim 15, wherein circulating the temperature-controlled fluid through the conduit layer includes circulating a cool fluid through the conduit layer for treating the wound, the wound selected from a heat burn, a friction burn, an electrical burn, and a radiation burn.

21. The method of claim 15, wherein circulating the temperature-controlled fluid through the conduit layer includes circulating a warm fluid through the conduit layer for treating the wound, the wound being frostbite.
